(19) United States
(12) Patent Application Publication
(10) Pub. No.: US 2025/0111920 A1
(43) Pub. Date: Apr. 3, 2025

(54) BODY POSITION CORRECTION

(71) Applicant: Nidhi O. Lawange, San Jose, CA (US)

(72) Inventor: Nidhi O. Lawange, San Jose, CA (US)

(21) Appl. No.: 18/479,906

(22) Filed: Oct. 3, 2023

(51) Int. Cl. G16H 20/30; A61B 5/00; G16H 30/40; G16H 50/20

(52) U.S. Cl. CPC G16H 20/30; A61B 5/4561; G16H 30/40; G16H 50/20

(57) ABSTRACT

Techniques are provided for body position correction. In one embodiment, the techniques involve receiving an image of a body, determining a body position based on the image, wherein the body position includes a movement, a pose, a posture, or a stance of the body, mapping markers to positions of the body, determining a body position angle, generating a correction angle of the body based on a comparison between the body position angle and a standard body position angle, and displaying a correction message.

### BACKGROUND

The present disclosure relates to body position correction, and more specifically, to remotely determining body positions and generating correction messages to adjust the body positions in relation to standard body positions.

Traditional body correction techniques can be performed remotely via a subject matter expert that visually inspects, and audibly corrects, a body pose via a video stream. However, the traditional body correction techniques can be hampered by poor network connections, or by faulty video equipment, which can produce low-quality or fragmented video streams. Further, subject matter experts may be unable to accurately determine correction angles from the video streams to effectuate the body position correction.

### SUMMARY

A method is provided according to one embodiment of the present disclosure. The method includes receiving an image of a body; determining a body position based on the image, wherein the body position includes a movement, a pose, a posture, or a stance of the body; mapping markers to positions of the body; determining a body position angle; generating a correction angle of the body based on a comparison between the body position angle and a standard body position angle; and displaying a correction message.

A system is provided according to one embodiment of the present disclosure. The system includes a processor; and memory or storage comprising an algorithm or computer instructions, which when executed by the processor, performs an operation that includes receiving an image of a body; determining a body position based on the image, wherein the body position includes a movement, a pose, a posture, or a stance of the body; mapping markers to positions of the body; determining a body position angle; generating a correction angle of the body based on a comparison between the body position angle and a standard body position angle; and displaying a correction message.

A computer-readable storage medium having computer-readable program code embodied therewith, the computer-readable program code executable by one or more computer processors to perform an operation, is provided according to one embodiment of the present disclosure. The operation includes receiving an image of a body; determining a body position based on the image, wherein the body position includes a movement, a pose, a posture, or a stance of the body; mapping markers to positions of the body; determining a body position angle; generating a correction angle of the body based on a comparison between the body position angle and a standard body position angle; and displaying a correction message.

### BRIEF DESCRIPTION OF THE DRAWINGS

FIG. 1 illustrates a body position correction environment, according to one embodiment.

FIG. 2 illustrates a computing environment, according to one embodiment.

FIG. 3 illustrates a flowchart of a method of generating a correction angle, according to one embodiment.

FIG. 4 illustrates a flowchart of a method of determining a body position angle, according to one embodiment.

### DETAILED DESCRIPTION

Embodiments of the present disclosure improve upon body correction systems by providing a body position correction (BPC) module that remotely determines body positions and generates correction angles and feedback to adjust the body positions in relation to standard body positions. In one embodiment, the BPC module uses a first machine learning model to identify a body position (e.g., a movement, a pose, a posture, a stance, or the like) of a body of a user that is included in an image. The BPC module can then use a second machine learning model to map markers to the body included in the image. The markers can be used to determine a body position angle in a region of interest of the body, and a correction angle can be determined using the body position angle and a standard body position angle. Afterwards, the BPC module can generate a correction message that includes the correction angle and correction feedback, and display the correction message to the user.

One benefit of the disclosed embodiments is to accurately determine correction angles to adjust a body position towards a standard body position. Further, embodiments of the present disclosure can provide a standardized approach to body position corrections, which is not dependent on the unique expertise of a given subject matter expert. Further, embodiments of the present disclosure allow for robust body position correction, which can operate despite poor network connections or faulty video equipment.

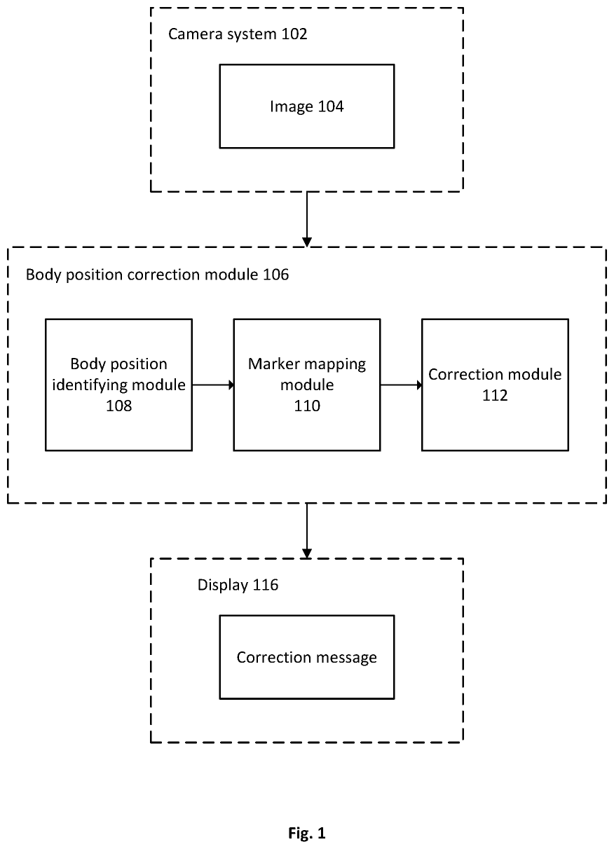

FIG. 1 illustrates a body position correction environment 100, according to one embodiment. In the illustrated embodiment, the body position correction environment 100 includes a camera system 102, an image 104, a body position correction (BPC) module 106 (which includes a body position identifying (BPI) module 108, a marker mapping (MM) module 110, and a correction module 112), a correction message 114, and a display 116.

In one embodiment, the camera system 102 captures an image 104 of a body position of a user. The body position can represent a movement, a pose, a posture, a stance, or the like, of the body included in the image. The camera system 102 can also record a video, and capture the image 104 (e.g., an image frame, or a picture) from the video. Although the image 104 is generally referenced as a single image, the image 104 may also include multiple images or image frames.

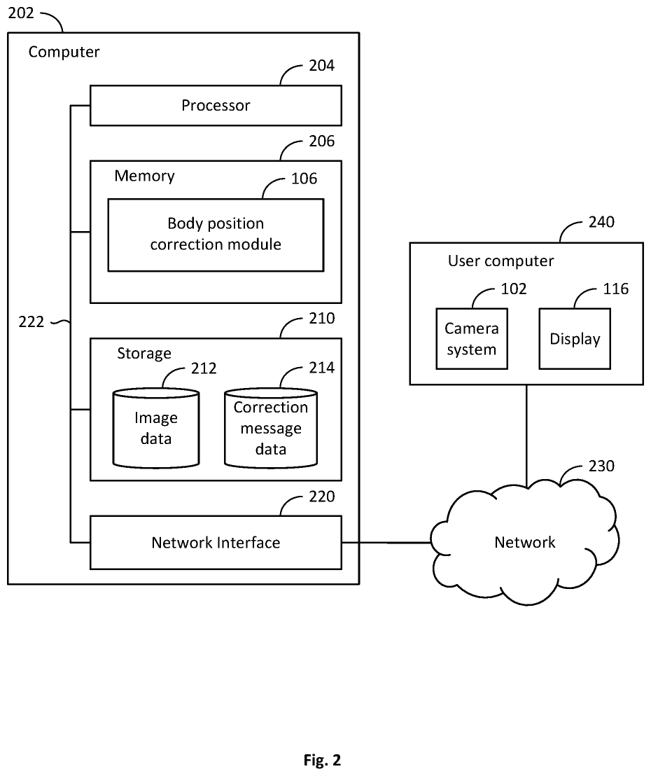

The image 104 can be input into the BPC module 106 to determine a correction message 114. This process is described in FIG. 2 below.

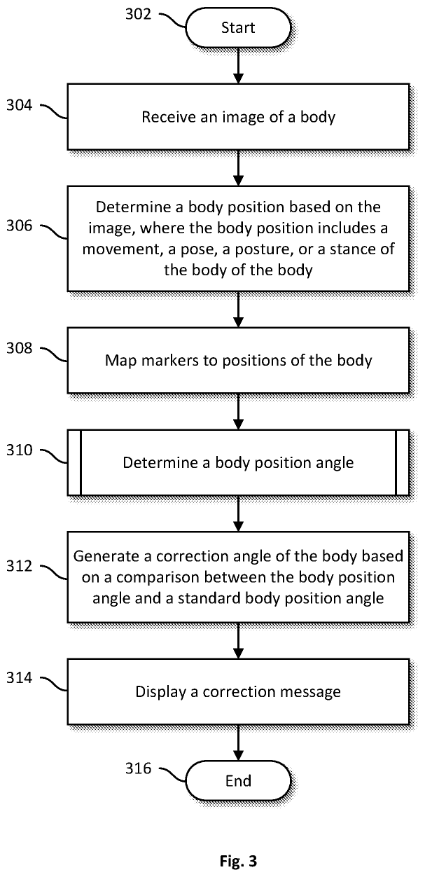

In one embodiment, the image 104 is processed by the BPI module 108 (a first machine learning model) to identify the body position of the body included in the image 104. For example, the BPI module 108 may identify the body position as a pose used in a ballet dance move, such as an arabesque, a passe, a sous-sous, a leap, or the like. This process is described further in FIG. 3 below.

Afterwards, the MM module 110 (a second machine learning model) can map markers to various positions of the body included in the image 104. In one embodiment, the markers are placed at the joints of the body. This process is described further in FIG. 3 below.

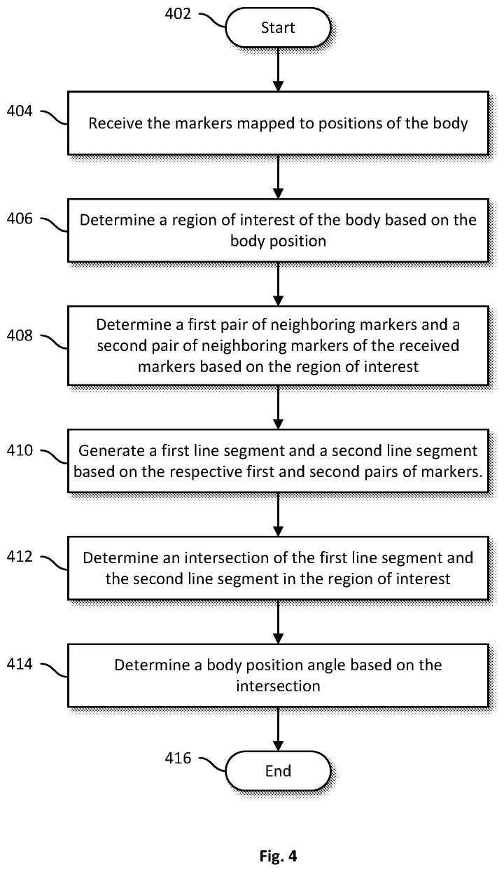

In one embodiment, the markers are input into the correction module 112, which uses the markers to generate a correction message 114 that can be used to correct the body position. The correction message 114 can include a correction angle and correction feedback. For example, the correction angle may identify an adjustment of a leg position that aligns the identified body position with a standard arabesque pose, while the correction feedback may offer a qualitative or quantitative rating based on the magnitude of the correction angle. This process is described further in FIGS. 3-4 below.

FIG. 2 illustrates a computing environment 200, according to one embodiment. In the illustrated embodiment, the computing environment 200 includes a computer 202, a network 230, and a user computer 240.

In one embodiment, the computer 202 includes a processor 204 that obtains instructions and data via a bus 222 from a memory 206 or storage 210. Not all components of the computer 202 are shown. The computer 202 can be under the control of an operating system (OS) suitable to perform or support the functions or processes disclosed herein. The processor 204 is a programmable logic device that performs instruction, logic, and mathematical processing, and may be representative of one or more CPUs. The processor may execute one or more algorithms, instruction sets, or applications in the memory 206 or storage 210 to perform the functions or processes described herein.

The memory 206 and storage 210 can be representative of hard-disk drives, solid state drives, flash memory devices, optical media, and the like. The storage 210 can also include structured storage (e.g., a database). In addition, the memory 206 and storage 210 may be considered to include memory physically located elsewhere. For example, the memory 206 and storage 210 may be physically located on another computer communicatively coupled to the computer 202 via the bus 222 or the network 230.

The computer 202 can be connected to other computers (e.g., controllers, distributed databases, servers, or webhosts), or the user computer 240 (which includes the camera system 102 and the display 116), via a network interface 220 and the network 230. Examples of the network 230 include electrical busses, physical transmission cables, optical transmission fibers, wireless transmissions mediums, routers, firewalls, switches, gateway computers, edge servers, a local area network, a wide area network, a wireless network, or the like. The network interface 220 may be any type of network communications device allowing the computer 202 to communicate with computers and other components of the computing environment 200 via the network 230.

In the illustrated embodiment, the memory 206 includes a body position correction (BPC) module 106. As previously described, the BPC module 106 can include a body position identifying (BPI) module 108, a marker mapping (MM) module 110, and a correction module 112. In one embodiment, the BPC module 106, and the modules included therein, represent one or more algorithms, instruction sets, software applications, or other computer-readable program code that can be executed by the processor 204 to perform the functions, operations, or processes described herein.

In one embodiment, the user computer 240 captures image data 212 (e.g., an image or video) of a user via the camera system 102. The image data 212 can be transferred to computer 202, and stored in the storage 210. The BPC module 106 can use the image data 212 to identify a body position of a body of the user. Further, the BPC module 106 can use the identification of the body position to map markers to the body, and then use the markers to generate correction message data 214 (e.g., a correction angle and correction feedback). The correction message data 214 can be stored in the storage 210 and shown on a display (not shown) of the computer 202. The correction message data 214 can also be transferred to the user computer 240, and shown on the display 116. Operation of the BPC module 106 is described in further detail in FIGS. 3-4 below.

FIG. 3 illustrates a flowchart of a method 300 of generating a correction angle, according to one embodiment. In one embodiment, the method 300 is performed by the body position correction module 106. As previously discussed, the body position correction (BPC) module 106 can include the body position identifying (BPI) module 108, the marker mapping (MM) module 110, and the correction module 112. The method 300 begins at block 302.

At block 304, the body position correction module 106 receives an image 104 of a body. In one embodiment, the BPC module 106 receives a video from the user computer 240, and captures the image 104 from the video.

At block 306, the body position identifying module 108 determines a body position based on the image 104, where the body position includes a movement, a pose, a posture, or a stance of the body. The BPI module 108 can include a machine learning model trained via a supervised learning process using images of standard and non-standard body positions.

A training image may be labeled as including a standard body position when a subject matter expert deems the body position shown in the training image to represent an ideal, exemplary, optimal, conventional, preferred, or normative body position. A training image may be labeled as including a non-standard body position when a subject matter expert deems the body position shown in the training image to represent a non-ideal, nonconforming, sub-optimal, non-conventional, non-preferred, or unconventional body position.

For example, a martial arts stance may consider a standard body position of a movement (e.g., a full-extension kick) to conform to a knee bend of 145-155 degrees for the hind leg, and little to no knee bend (e.g., 176-180 degrees) for the extended leg. A non-standard body position may include any knee bends outside of the knee bend ranges of the standard body position.

In one embodiment, the BPI module 108 is a convolutional neural network (CNN) that receives the image 104, detects edges of the body shown in the image 104, compares the edges to known edge positions of the body learned from the training data, and outputs a probability distribution of identifications of the body position included in the image 104. The greatest probability distribution can be compared to an identification threshold to determine whether the image 104 is transferred to the MM module 110. The identification threshold can represent a configurable accuracy confidence value of the identification. In one embodiment, the identification threshold is set to a value of at least 65%. In another embodiment, the identification threshold is set to a value of at least 80%.

Upon determining that the greatest probability distribution does not exceed the identification threshold, the BPI module 108 can disregard the identification results, and perform another identification, or await another input image. However, upon determining that the greatest probability distribution does exceed the identification threshold, the BPI module 108 transfers the image 104 to the MM module 110, and the method 300 proceeds to block 308.

At block 308, the marker mapping module 110 maps markers to positions of the body. The MM module 110 can include a machine learning model trained to perform a pose estimation process. In one embodiment, the pose estimation process involves determining a position or orientation of the body shown in the image 104 in a 2-dimensional or 3-dimensional coordinate system.

In one embodiment, the MM module 110 performs a 3-dimensional pose estimation by establishing x, y, and z axes of the image, and then determining (x, y, z) coordinates of each joint of the body shown in the image 104.

Afterwards, the MM module 110 can use the coordinates to map a marker to each joint of the body. In one embodiment, each marker also includes a joint identifier (e.g., an “right elbow joint”).

At block 310, the correction module 112 performs a method 400 to determine a body position angle. In one embodiment, the body position angle represents an angle used to characterize the body position of the body shown in the image 104. The method 400 is described further in FIG. 4 below.

At block 312, the correction module 112, generates a correction angle of the body based on a comparison between the body position angle and a standard body position angle of the body position. In one embodiment, the correction angle represents an angle by which the body position that can be adjusted to reflect the standard body position.

The standard body position angle can represent an angle that characterizes a standard body position. Put differently, the standard body position angle can represent an angle of a corresponding standard body position that is used to determine an extent to which a body position conforms to the standard body position. In one embodiment, the standard body position angle is identified from a predetermined mapping between body positions and corresponding standard body position angles, as determined by a subject matter expert. The predetermined mappings may be stored in a database of the storage 210, or on another computer connected to the network 230.

Continuing the previous example, the standard body position angle of the full-extension kick includes a knee bend of 145-155 degrees for the hind leg. Assuming that the body shown in the image 104 has a knee bend of 165 degrees for the hind leg (i.e., the method 400 determines that the body position angle is 165 degrees), the correction module 112 can determine the correction angle to be within the range of 10-20 degrees. Hence, adjusting the body position angle of 165 degrees by the correction angle of 10-20 degrees can match the standard body position angle of 145-155 degrees.

At block 314, the correction module 112 displays a correction message. In one embodiment, the correction message includes the correction angle and correction feedback. The correction feedback may describe a positive sentiment, a negative sentiment, a rating, instructions to correct the body position, or the like.

Continuing the previous example, upon determining the correction angle of 10-20 degrees for the hind leg, the correction module can generate a message such as, “Good kick; try bending the knee of your hind leg by 10-20 degrees; there are no adjustments needed for your front leg.” The method 300 ends at block 316.

FIG. 4 illustrates a flowchart of a method 400 of determining a body position angle, according to one embodiment. The method 400 begins at block 402.

At block 404, the correction module 112 receives the markers mapped to positions of the body. As previously discussed, the markers may be placed at the joints of the body shown in the image 104. Further, the markers can include (x, y, z) coordinates of a 3-dimensional coordinate system of the image 104.

At block 406, the correction module 112 determines a region of interest of the body based on the body position. In one embodiment, the region of interest represents a set of the markers that corresponds to the body position.

Predetermined mappings between the body positions and sets of markers may be stored in a database of the storage 210, or on another computer connected to the network 230. The correction module 112 can determine the region of interest using the predetermined mappings. For example, assuming that the body position is a ballet leap, the predetermined mappings may include markers positioned at a center of the hips, at a left knee joint, and at a right knee joint.

At block 408, the correction module 112 determines a first pair of neighboring markers and a second pair of neighboring markers of the received markers based on the region of interest. In one embodiment, the neighboring markers are determined by traversing continuous, connected parts of the anatomy of the body.

Continuing the previous example, the first pair of neighboring markers can be the markers positioned at the center of the hips and the left knee joint. The second pair of neighboring markers can be the markers positioned at the center of the hips and the right knee joint.

At block 410, the correction module 112 generates a first line segment and a second line segment based on the respective first and second pairs of markers. In one embodiment, the correction module 112 can determine a given line segment as the distance between the (x, y, z) coordinates of a pair of markers of the region of interest.

Continuing the previous example, the first line segment can be determined as the distance between the coordinates of the markers positioned at the center of the hips and the left knee joint. The second line segment can be determined as the distance between the coordinates of the markers positioned at the center of the hips and the right knee joint.

At block 412, the correction module 112 determines an intersection of the first line segment and the second line segment in the region of interest. In one embodiment, the correction module 112 determines the slopes of the first line segment and the second line segment, and determines the intersection based on the slopes.

At block 414, the correction module 112 determines a body position angle based on the intersection. In one embodiment, the correction module 112 determines the body position angle at the intersection as the difference between the arc-tangents of the slopes of the first and second line segments. The body position angle may be determined with respect to 2-dimensional axes.

Continuing the previous example, the body position angle can represent the angle formed by the left knee joint, the center of the hips, and the right knee joint of the body shown in the image 104. The method 400 ends at block 414.

While the foregoing is directed to embodiments of the present invention, other and further embodiments of the invention may be devised without departing from the basic scope thereof, and the scope thereof is determined by the claims that follow.

1. A method comprising:
receiving an image of a body;
determining a body position based on the image, wherein the body position includes a movement, a pose, a posture, or a stance of the body;
mapping markers to positions of the body;
determining a body position angle;
generating a correction angle of the body based on a comparison between the body position angle and a standard body position angle; and
displaying a correction message.

2. The method of claim 1, wherein the determining the body position involves:
outputting, via a first machine learning model, a probability distribution of identifications of the body position based on the image; and
upon determining that the probability distribution exceeds an identification threshold, transferring the image to a second machine learning model, wherein the identification threshold represents a configurable accuracy confidence value of at least 65%.

3. The method of claim 1, wherein determining the body position angle involves:
receiving the markers mapped to the positions of the body;
determining a region of interest of the body based on the body position;
determining a first pair of neighboring markers and a second pair of neighboring markers of the received markers based on the region of interest;
generating a first line segment and a second line segment based on the respective first and second pairs of markers;
determining an intersection of the first line segment and the second line segment in the region of interest; and
determining a body position angle based on the intersection.

4. The method of claim 3, wherein the region of interest represents a set of markers that correspond to the body position; and
wherein the region of interest is determined based on a predetermined mapping of the body position to a region of interest.

5. The method of claim 1, wherein the markers are mapped to the positions of the body via a second machine learning model;
wherein the positions of the body represent joints of the body;
wherein the markers include coordinates on a 3-dimensional coordinate system of the image; and
wherein the markers include a joint identifier.

6. The method of claim 1, wherein the correction angle represents an angle by which the body position can be adjusted to reflect a standard body position;
wherein the correction message includes the correction angle and correction feedback; and
wherein the correction feedback describes a positive sentiment, a negative sentiment, a rating, or instructions to correct the body position.

7. The method of claim 6, wherein the standard body position represents an exemplary or conventional body position.

8. A system, comprising:
a processor; and
memory or storage comprising an algorithm or computer instructions, which when executed by the processor, performs an operation comprising:
receiving an image of a body;
determining a body position based on the image, wherein the body position includes a movement, a pose, a posture, or a stance of the body;
mapping markers to positions of the body;
determining a body position angle;
generating a correction angle of the body based on a comparison between the body position angle and a standard body position angle; and
displaying a correction message.

9. The system of claim 8, wherein the determining the body position involves:
outputting, via a first machine learning model, a probability distribution of identifications of the body position based on the image; and
upon determining that the probability distribution exceeds an identification threshold, transferring the image to a second machine learning model, wherein the identification threshold represents a configurable accuracy confidence value of at least 65%.

10. The system of claim 8, wherein determining the body position angle involves:
receiving the markers mapped to the positions of the body;
determining a region of interest of the body based on the body position;
determining a first pair of neighboring markers and a second pair of neighboring markers of the received markers based on the region of interest;
generating a first line segment and a second line segment based on the respective first and second pairs of markers;
determining an intersection of the first line segment and the second line segment in the region of interest; and
determining a body position angle based on the intersection.

11. The system of claim 10, wherein the region of interest represents a set of markers that correspond to the body position; and
wherein the region of interest is determined based on a predetermined mapping of the body position to a region of interest.

12. The system of claim 8, wherein the markers are mapped to the positions of the body via a second machine learning model;
wherein the positions of the body represent joints of the body;
wherein the markers include coordinates on a 3-dimensional coordinate system of the image; and
wherein the markers include a joint identifier.

13. The system of claim 8, wherein the correction angle represents an angle by which the body position can be adjusted to reflect a standard body position;
wherein the correction message includes the correction angle and correction feedback; and
wherein the correction feedback describes a positive sentiment, a negative sentiment, a rating, or instructions to correct the body position.

14. The system of claim 13, wherein the standard body position represents an exemplary or conventional body position.

15. A computer-readable storage medium having a computer-readable program code embodied therewith, the computer-readable program code executable by one or more computer processors to perform an operation comprising:
receiving an image of a body;
determining a body position based on the image, wherein the body position includes a movement, a pose, a posture, or a stance of the body;
mapping markers to positions of the body;
determining a body position angle;
generating a correction angle of the body based on a comparison between the body position angle and a standard body position angle; and
displaying a correction message.

16. The computer-readable storage medium of claim 15, wherein the determining the body position involves:
outputting, via a first machine learning model, a probability distribution of identifications of the body position based on the image; and
upon determining that the probability distribution exceeds an identification threshold, transferring the image to a second machine learning model, wherein the identification threshold represents a configurable accuracy confidence value of at least 65%.

17. The computer-readable storage medium of claim 15, wherein determining the body position angle involves:
receiving the markers mapped to the positions of the body;
determining a region of interest of the body based on the body position;
determining a first pair of neighboring markers and a second pair of neighboring markers of the received markers based on the region of interest;
generating a first line segment and a second line segment based on the respective first and second pairs of markers;
determining an intersection of the first line segment and the second line segment in the region of interest; and
determining a body position angle based on the intersection.

18. The computer-readable storage medium of claim 17, wherein the region of interest represents a set of markers that correspond to the body position; and
wherein the region of interest is determined based on a predetermined mapping of the body position to a region of interest.

19. The computer-readable storage medium of claim 15, wherein the markers are mapped to the positions of the body via a second machine learning model;
wherein the positions of the body represent joints of the body;
wherein the markers include coordinates on a 3-dimensional coordinate system of the image; and
wherein the markers include a joint identifier.

20. The computer-readable storage medium of claim 15, wherein the correction angle represents an angle by which the body position can be adjusted to reflect a standard body position;
wherein the correction message includes the correction angle and correction feedback;
wherein the correction feedback describes a positive sentiment, a negative sentiment, a rating, or instructions to correct the body position; and
wherein the standard body position represents an exemplary or conventional body position.
